(19) United States
(12) Patent Application Publication
(10) Pub. No.: US 2007/0275082 A1
(43) Pub. Date: Nov. 29, 2007

(54) Preparation Method for Sustained Release Microspheres Using a Dual-Feed Nozzle

(76) Inventors: Hee Yong Lee, Daejeon (KR); Sung Kyu Kim, Daejeon (KR); Jung Soo Kim, Daejeon (KR); Young Hwan Jung, Daejeon (KR); Jung In Kim, Daejeon (KR); Ho Il Choi, Daejeon (KR); Seung Gu Chang, Daejeon (KR); Kee Don Park, Daejeon (KR)

Correspondence Address: MEDLEN & CARROLL, LLP, 101 HOWARD STREET, SUITE 350, SAN FRANCISCO, CA, 94105, US

(21) Appl. No.: 10/570,564

(22) PCT Filed: Sep. 3, 2004
(86) PCT No.: PCT/KR04/02241

§ 371 Date: Aug. 17, 2007

(30) Foreign Application Priority Data
Sep. 4, 2003 (KR) 10-2003-0061943

(51) Int. Cl. A61K 38/09; A61K 9/16

(52) U.S. Cl. 424/497; 424/490; 424/498; 514/12; 514/2; 514/9; 514/15; 264/4

(57) ABSTRACT

Disclosed is a method of preparing sustained release microspheres by spray-drying liquids with different compositions for preparation the sustained release microspheres through an ultrasonic dual-feed nozzle. Unlike conventional methods of preparing sustained release microspheres by spray-drying a single liquid containing a biodegradable polymer, a drug, an additive and a solvent through a single-feed nozzle, the present method is characterized by simultaneously spray-drying two liquids with different compositions for preparation of the sustained release microspheres respectively through internal and external channels of an ultrasonic dual-feed nozzle to coat sprayed droplets through the internal channel with other sprayed droplets through the external channel. The present method is effective in achieving a low initial release and a desired continuous release.

### TECHNICAL FIELD

The present invention relates to a method of preparing sustained release microspheres, which is based on encapsulating a drug in a biodegradable polymer carrier by spray-drying using an ultrasonic dual-feed nozzle to achieve sustained release of the drug.

### BACKGROUND ART

Drugs having a relatively short half-life, including peptides or proteins useful as pharmaceutical preparations, need to be frequently administered to be maintained at effective concentrations in the blood. In this regard, new pharmaceutical formulations have been developed to enhance the convenience for patients and improve therapeutic efficacy and safety by maintaining blood drug levels within a therapeutically effective range. A preferred representative example is an injectable sustained release microsphere formulation, which contains a drug encapsulated in a biodegradable polymer carrier and provides sustained release of the drug at effective concentrations.

Typically, sustained release microsphere formulations containing peptide or protein drugs are manufactured by phase separation, double emulsion solvent extraction and evaporation and spray-drying methods. Generally, from the sustained release microspheres, the initial release of a drug must not be at high levels but at suitable levels, and a continuous release of the drug must be also suitably achieved. However, when sustained release microspheres are manufactured by the aforementioned conventional methods, in most cases, they release encapsulated drugs at a high rate at the initial phase and do not deliver the drug at a constant rate for a long period of time. Even when several preparation parameters are changed to reduce the initial release of a drug, the drug is not completely released even after a predetermined period, or the drug is often not released at the initial phase. In particular, in the case of water-soluble drugs, such as peptides or proteins, the aforementioned technical problems are not easily solved. When encapsulated in sustained release microspheres by the aforementioned conventional methods, water-soluble drugs are not evenly distributed in microsphere matrices but mainly distributed on the surface of the microsphere matrices, leading to a high initial release rate. In addition, when protein drugs with relatively high molecular weights are encapsulated in microspheres using protein microparticles rather than protein solutions to minimize their denaturation, the aforementioned technical problems become more difficult to solve.

These problems can be solved by a method disclosed in U.S. Pat. No. 6,120,787, which is based on preparing primary microparticles entrapping a drug and coating the primary microparticle core with a different biodegradable polymer. In detail, this method comprises preparing core particles entrapping a protein drug therein using starch, drying the core particles, and coating the core particles with a biodegradable polymer dissolved or dispersed in an organic solvent in a fluidized bed. Since the drug-entrapping core particles are coated with a different biodegradable polymer, the initial release of the trapped drug is reduced. However, in a test of the drug release, according to the degree of coating, the entrapped drug was not released at the initial phase but was released after a predetermined period. In addition, because a currently available fluidized bed coating apparatus commercially or technically requires a minimum production scale of several tens of grams, it has limited applications for expensive drugs. Further, this method is problematic upon industrial application because it provides a complicated two-step process including preparing core particles and coating the core particles.

An alternative method is a one-step method of preparing multi-layered polymeric microspheres using polymers, as reported by Mathiowitz et al. in U.S. Pat. No. 5,912,017. The polymers used in preparing microspheres are biodegradable or non-biodegradable and have different surface tension or interfacial tension properties. With the one-step method based on double emulsion solvent extraction and evaporation, multi-layered microspheres were successfully manufactured. However, the one-step method has a limitation in general applications because not all polymers applicable to drug delivery systems,- except for those illustrated in the embodiments of the patent, have different surface tension or interfacial tension properties from each other. In addition, it is expected to be preferable to entrap a physiologically active substance in the core of a microsphere. However, the one-step method makes it difficult to locate most drugs in a specific region, and preferably the core, of a microsphere.

Despite many previous studies, there is a need for a novel method of preparing sustained release microspheres entrapping peptide or protein drugs, which is capable of inhibiting a high initial release of the drugs and releasing the drugs at a constant rate for a long period of time, as well as providing a simple manufacturing process.

Therefore, the present invention aims to provide a method of preparing sustained release microspheres, which is capable of easily achieving a desired release pattern of drugs by a one-step process to avoid a high initial drug release and a drug release that sharply decreases or increases in the course of time.

Leading to the present invention, the intensive and thorough research, conducted by the present inventors with an aim to improve the disadvantages of conventional sustained release microsphere formulations, resulted in the establishment of a novel one-step process, which is based on simultaneously spray-drying two different liquids containing a biodegradable polymer, a drug, an additive and a solvent with different types or contents or both of the components through a single dual-feed nozzle comprising internal and external channels to produce double-layered microspheres where droplets sprayed through the internal channel are coated with other droplets sprayed through the. external channel, and resulted in the finding that, from the microspheres, the drug release is controlled for a desired period of time without a high initial release.

### DISCLOSURE OF THE INVENTION

The present invention provides a method of preparing sustained release microspheres encapsulating a drug in a biodegradable polymer carrier, comprising (a) preparing two different liquids for preparation of the sustained release microspheres containing a biodegradable polymer, a drug, an additive and a solvent with different compositions for one or more of the components; (b) simultaneously spraying the two different liquids respectively through internal and external channels of an ultrasonic dual-feed nozzle, wherein one liquid is supplied through the internal channel and another liquid is supplied through the external channel; and (c) evaporating the solvent using dry air to dry sprayed droplets.

In the present method, the liquid supplied to the external channel of the dual-feed nozzle preferably does not contain water.

The biodegradable polymer is preferably one or more selected from the group consisting of polyesters, which are exemplified by polylactide (PLA), polyglycolide (PGA), and their copolymer, poly(lactide-co-glycolide) (PLGA) or its star polymer, poly(lactide-co-glycolide)-glucose (PLGA-glucose), polyorthoesters, polyanhydrides, polyamino acids, polyhydroxybutyric acid, polycaprolactone, polyalkylcarbonate, lipids, fatty acids and waxes, and is most preferably selected from among polylactide and poly(lactide-co-glycolide).

In addition, the drug is preferably selected from among peptides and proteins.

### BRIEF DESCRIPTION OF THE DRAWING

FIG. 1 shows the results of in vitro drug release tests of sustained release microspheres prepared according to the procedures of Example 1 and Comparative Examples 1 and 2 of the present invention.

### DETAILED DESCRIPTION OF THE INVENTION

The present invention provides a method of preparing sustained release microspheres, comprising suspending, emulsifying or, more preferably, dissolving a drug or an additive to be encapsulated with identical or different concentrations in solutions of different types or concentrations of biodegradable polymers, and supplying the resulting liquids to a spray drier through a single dual-feed nozzle to produce double-layered sustained release microspheres including a core coated with a film having different compositions.

In detail, as the liquids supplied to the dual-feed nozzle, two or more different liquids for preparation of the sustained release microspheres are used, which contain a biodegradable polymer, a drug, an additive and a solvent with different compositions for one or more of the components, and are preferably in a solution form. When the drug is a peptide, the liquids containing the peptide preferably do not contain water and is selected from acetic acid, formic acid and mixtures thereof. The acetic acid is preferably glacial acetic acid. Especially preferably, the liquid supplied to the external channel of the dual-feed nozzle does not contain water.

The term “biodegradable polymer”, as used herein, includes synthetic polymers, which are exemplified by polyesters, such as polylactide (PLA), polyglycolide (PGA) and their copolymer, poly(lactide-co-glycolide) (PLGA) or its star polymer, poly(lactide-co-glycolide)-glucose (PLGA-glucose), polyorthoesters, polyanhydrides, polyamino acids, polyhydroxybutyric acid, polycaprolactone and polyalkylcarbonate, and naturally occurring lipids including fats, fatty acids, waxes and their derivatives. The above examples of the biodegradable polymer are provided only to illustrate the present invention, and the present invention is not limited to them.

In particular, among the aforementioned biodegradable polymers, the polyesters, such as PLA, PGA or PLGA, are approved to be biocompatible and safe to the body because they are metabolized in vivo to harmless lactic acid and glycolic acid by hydrolysis. The degradation of the polyesters may be controlled at various rates according to the molecular weight, the ratio of the two monomers, the hydrophilicity, and the like, for various durations ranging from a short period of one to two weeks to a long period of one to two years. The polyesters are polymeric substances that have been approved for use in humans in several tens of countries, including by the U.S. Food and Drug Administration (FDA), and commercialized. Therefore, the polyesters may be preferably used in the present invention. In particular, the polyesters such as PLGA or PLA may be preferably used in the present invention.

The release pattern of a drug from sustained release microspheres greatly depends on hydration rate and degradation rate of the polymer used, affinity of the drug to the polymer, surface or internal configuration of the microspheres, and the like. The hydration and degradation rates of the polymer depend on hydrophilicity thereof. In case of PLGA or PIA polymers, polymers having free carboxyl end groups (e.g., RG502H, RG503H, RG504H, R202H, R203H, etc., which are produced by Boehringer Ingelheim) are more rapidly hydrated due to their high hydrophilicity than polymers having free carboxyl end groups substituted with alkyl groups such as dodecyl groups (e.g., RG502, RG503, RG504, R202, R203, etc., which are produced by Boehringer Ingelheim), and, thus, are rapidly degraded in vivo. In addition, the degradation rate of the polymer greatly depends on the molecular weight and the ratio of the lactic acid residues to the glycolic acid residues. PLGA polymers including lactic acid residues and glycolic acid residues at a ratio of 50:50 are most quickly degraded, which are exemplified by RG502H, RG502 and RG503H, and, among the PLGA polymers containing lactic acid residues to glycolic acid residues at an equal content, low molecular weight polymers are more quickly degraded. As polymers have higher lactide contents, such as RG7525(H) or RG8515(H), they are degraded at slower rates. Thus, among polymers with an identical molecular weight, PLA polymers consisting of only lactic acids, such as R202(H) or R203(H), are most slowly degraded. With regard to the degradation rate of the polymer and other factors, PLGA polymers including lactic acid residues and glycolic acids at a ratio of 50:50 are used when drugs are desired to be released within one month. Polymers including 75% or 100% lactic acid residues are used mainly when drugs are desired to be released for two to three months or for a longer period of time.

The drug applicable in the present invention includes all drugs in various forms, such as peptides, proteins and synthetic organic compounds. The drugs may have various biological activities, for example, serving as anticancer agents, antibiotics, analgesics, antiinflammatory agents, sedatives, antiulcer agents, antidepressants, antiallergenic agents, therapeutic agents against diabetes mellitus, therapeutic agents against hyperlipidemia, antituberculous agents, hormonal agents, anesthetics, bone metabolic agents, immunomodulators, angiogenesis regulators, contraceptives, and vitamin-like agents, but are not limited to them.

Biologically active peptide and protein drugs are preferably used in the present invention. Especially preferred biologically active peptides are biologically active peptides of 2 to 60 amino acid residues, salts thereof or analogues thereof. Examples of peptides composed of 5 or fewer amino acid residues in length include glutathione, homoglutathione, endomorphin, thymopoietin and enkephalin. Examples of peptides composed of 10 or fewer amino acid residues include growth hormone release peptide-2 and -6 (GHRP-2 and -6), octreotide, carbetocin, oxytocin, cholecystokinin, vasopressin, bradykinin, delta sleep-inducing peptide, angiotensin I, II and III, neurokinin A and B, neuromedin B, triptorelin, leuprolide, goserelin, nafarelin, buserelin, histerelin, antide, argtide, orntide, and cetrorelix. Examples of peptides composed of 20 or fewer amino acid residues include hirudin, alloferin 1 and 2, IGF-1 analogues, cortistain-17, dynorphin A and B, α-endorphin, γ-endorphin, gastrin, guanylin, uroguanylin, and substance P. Examples of peptides composed of 30 or fewer amino acids include defensin 1 and 2, gastrin releasing peptide, secretin, endothelin, and glucagon-like peptide-2. Examples of peptides composed of 40 or fewer amino acid residues include ceropin A, B and P1, pancreatic polypeptide, amylin, calcitonin, calcitonin gene related peptide, β-endorphin, and Big endothelin-1. Examples of peptides composed of 60 or fewer amino acid residues include corticotropin releasing factor, growth hormone releasing factor (GRF), adrenomedullin, C-type natriuretic peptide, and insulin. More preferred are biologically active peptides of 3 to 30 amino acid residues in length, and most preferred are biologically active peptides of 5 to 20 amino acid residues in length.

In embodiments of the present invention, the polyesters such as PLGA are used as the biodegradable polymer, and peptide drugs, such as octreotide and luteinizing hormone releasing hormone (LHRH) analogs, are mainly used. The embodiments demonstrate that protein drugs are suitable for the purpose of the present invention. When octreotide or LHRH analogs are to be used, their salts of acetate are more preferred.

The LHRH analogues refer to peptides that, when administered to the body, inhibit the secretion of LH by the pituitary gland (in case of LHRH agonists, the secretion of LH is stimulated in the early phase but is inhibited upon continuous release), leading to inhibition of secretion of testosterone and estrogen, and that, due to this action, have therapeutic efficacy on hormone-dependent diseases, such as prostatic cancer, endometriosis and uterine myoma. Non-limiting examples of the LHRH analogs include LHRH agonists, such as triptorelin, leuprolide, goserelin, nafarelin, buserelin, histerelin and salts thereof, and LHRH antagonists, such as antide, argtide, orntide, cetrorelix and salts thereof.

Octreotide, which is a somatostatin variant, is a peptide drug consisting of eight amino acids. Octreotide has stronger affinity to somatostatin receptors than the naturally occurring somatostatin, and, thus, is more effective in inhibiting the release of growth hormone, glucagons and insulin than somatostatin. In addition, octreotide suppresses the release of luteinizing hormone (LH) by gonadotropin-releasing hormone, decreases splanchnic blood flow, and inhibits the release of serotonin, gastrin, vasoactive intestinal peptide (VIP), secretin, motilin, and the like. By virtue of these pharmacologic actions, octreotide has been used to treat the symptoms associated with metastatic carcinoid tumors (flushing and diarrhea) and vasoactive intestinal peptide (VIP)-secreting adenomas (watery diarrhea). Also, octreotide has been used to reduce the release of growth hormone and insulin-like growth hormone in acromegaly patients.

The additive applicable in the liquid for the preparation of sustained release microspheres of the present invention may include sucrose, trehalose, maltose, mannitol, lactose, mannose, cyclodextrin, dextran, polyethyleneglycol, polyvinylpyrrolidone, albumin, surfactants, amino acids, lactic acid, and inorganic salts. The solvent applicable in the fluid for the preparation of sustained release microspheres of the present invention may include glacial acetic acid, formic acid, acetonitrile, ethylacetate, acetone, methylethylketone, methylene chloride, chloroform, ethanol, and methanol.

The two or more liquids as prepared above are supplied to a spray drier through an ultrasonic dual-feed nozzle. Preheated and dried air at high temperature is supplied to an upper portion of the spray drier, to which the ultrasonic dual-feed nozzle is installed, and the liquids sprayed from the nozzle are dried and recovered in the form of microspheres.

When microspheres are prepared by a spray-drying method, the release rate of a drug greatly depends on the compositions of solutions to be sprayed, such as composition or content of a biodegradable polymer, drug content, additive type or content and solvent amount. In addition to the above processing parameters, other parameters affecting the size or morphology of microspheres may be employed to control the release rate of drugs, which include methods of spraying the solutions (for example, spraying methods using pressure, air and ultrasonic wave), spray nozzle type, supply rate of solutions to be sprayed, size of sprayed droplets (for example, in case of using the air spraying method using air, amount of air supplied to the spray nozzle; in case of using the ultrasonic spraying method, frequencies of ultrasonic waves), supplied amount of dry air, and supply rate and temperature of the dry air.

Since the present invention aims to prepare a microsphere formulation capable of achieving a greatly decreased initial release and a continuous release at a constant rate in comparison with conventional microspheres prepared using a single-feed nozzle, it will be apparent to those skilled in the art that preparation parameters except for the composition and supply method of the spray liquids are suitably controlled according to the purpose of the present invention.

The terms “dual-feed nozzle” and “single-feed nozzle”, as used herein, are classified according to the number of liquids supplied to a spray nozzle, that is, the number of liquids containing a biodegradable polymer, a drug, an additive and a solvent. To a dual-feed nozzle, liquids with different compositions are supplied through different channels. To a single-feed nozzle, liquids with identical compositions are supplied. The “dual-feed nozzle” is composed of an internal channel and an external channel, to which liquids with different compositions are supplied. The term “dual-feed nozzle”, as used herein, has a meaning different from a typically used term “two-fluid nozzle”. The two-fluid nozzle is also composed of an internal channel and an external channel. Upon using the two-fluid nozzle, a spray liquid (liquid-1) is typically sprayed through the internal channel, while air or gas is supplied to the external channel. Thus, the two-fluid nozzle corresponds to the single-feed nozzle.

A conventional method of preparing microspheres by spray-drying using two nozzles is disclosed in U.S. Pat. No. 5,622,657. To improve the disadvantages of conventional methods including dispersing microspheres in a dispersing agent solution and drying the resulting dispersion to avoid microspheres prepared by spray drying adhering to each other or aggregating, the cited patent provides a process for the production of a microparticle preparation, comprising spraying a solution of a polymer containing a biologically active substance and an aqueous solution of an agent for preventing aggregation of microparticles separately from different nozzles at the same time and contacting them with each other in a spray dryer to produce polymeric microparticles which contain a drug and are coated with a film of the agent for preventing aggregation of the microparticles. In the cited patent, the aqueous solution of an aggregation-preventing agent is sprayed through a different nozzle to prevent aggregation of polymeric microspheres. In contrast, the present invention is characterized by simultaneously spraying liquids with different compositions containing a biodegradable polymer for preparation of sustained release microspheres respectively through internal and external channels of a single dual-feed nozzle in a suitable ratio, thereby making it possible to reduce the initial release of a drug and to achieve a desired continuous release of the drug.

The dual-feed nozzle used in the present invention is a dual-feed microencapsulation nozzle. In one embodiment, the dual-feed nozzle used in examples is connected to an ultrasonic generator of 25 kHz, thereby generating small droplets 50-100 μm in diameter, on average. The nozzle includes two channels where liquids are individually supplied. For example, the nozzle includes a channel having an inner diameter of 1 mm and another channel having an inner diameter smaller than the above channel and being inserted into the above channel, for example, a microtube of 0.5 mm. Thus, when two liquids are simultaneously supplied through corresponding channels to a spray drier and sprayed through the channels in the spray drier, the liquid (liquid A) sprayed through the internal channel forms an inner core of polymeric microspheres, and the liquid (liquid B) sprayed through the external channel forms a film coating the inner core at the same time. Spraying is conducted in a dry atmosphere.

The present inventors prepared microspheres using two polymer types having different physicochemical properties, selected from among several types of a biodegradable polymer, PLGA, by simultaneously spraying liquid A containing octreotide and PLGA and liquid B having an equal concentration of another type of PLGA respectively through internal and external channels of a dual-feed nozzle. Also, the initial release and continuous release of a drug from microspheres was found to be controlled by varying the type and ratio of polymers, the content of a drug and the ratio of an additive, thereby leading to the present invention. PLGA and PLA used in embodiments of the present invention all were purchased from Boehringer Ingelheim. In the practice of the present invention, a dual-feed nozzle was used to supply two liquids with different compositions for preparation of sustained release microspheres to a spray drier. However, it will be apparent to those skilled in the art that the initial release and release pattern of a drug can be controlled by simultaneously spray-drying more than two liquids using a multi-feed nozzle.

The drug-loaded polymeric microspheres of the present invention may be administered as they are, as an implant, or may be formulated into various pharmaceutical dosage forms. In the latter case, the microspheres may be used as a raw material for various pharmaceutical formulations. Examples of the pharmaceutical formulations include injectable preparations, preparations for oral administration (e.g., powders, granules, capsules, tablets, etc.), preparations for intranasal administration, and suppositories (e.g., suppositories for intrarectal administration, suppositories for intravaginal administration). These preparations can be prepared according to the methods well known in the art.

A better understanding of the present invention may be obtained through the following examples which are set forth to illustrate, but are not to be construed as the limit of the present invention.

### EXAMPLE 1

### Preparation of Octreotide-Loaded PLGA Microspheres using a Dual-Feed Nozzle

Solutions A and B, to be supplied to a spray drier respectively through internal and external channels of a dual-feed nozzle, were prepared using biodegradable polymers and a drug. RG502H and RG504H biodegradable polymers were used, and octreotide was used as the drug. Microspheres were prepared to contain the drug in a final concentration of 2 wt % according to the following procedure.

Solution A, to be supplied through the internal channel of a dual-feed nozzle, was prepared by homogeneously dissolving 0.5 g of the biodegradable polymer RG502H and 20 mg of octreotide in 10 ml of glacial acetic acid. Solution B, to be supplied through the external channel of the dual-feed nozzle, was prepared by homogeneously dissolving 0.5 g of the biodegradable polymer RG504H in 10 ml of glacial acetic acid. The two solutions were supplied to a spray drier at a flow rate of 1 ml/min respectively through internal and external channels of an ultrasonic dual-feed nozzle (Sono-Tek, 8700-25MS), sprayed in the spray drier (Kwangjin Corporation, Korea), and dried with dry air at 105° C., thereby yielding microspheres. The final microspheres were 28.8 μm in diameter, on average.

### COMPARATIVE EXAMPLE 1

### Preparation of Octreotide-Loaded RG502H Microspheres using a Single-Feed Nozzle

Microspheres were prepared to contain octreotide as a drug in a final concentration of 2 wt % using a biodegradable polymer, RG502H, according to the following procedure.

1 g of RG502H and 20 mg of octreotide were homogeneously dissolved in 20 ml of glacial acetic acid. The resulting solution was supplied to a spray drier at a flow rate of 2 ml/min through an ultrasonic nozzle (Sono-Tek, 8700-60 MS) that is a general single-feed type, sprayed in the spray drier (Kwangjin Corporation, Korea), and dried with dry air at 105° C., thereby yielding microspheres. The final microspheres were 27.5 μm in diameter, on average.

### COMPARATIVE EXAMPLE 2

### Preparation of Octreotide-Loaded RG504H Microspheres using a Single-Feed Nozzle

Microspheres were prepared to contain octreotide as a drug in a final concentration of 2 wt % using a biodegradable polymer, RG504H, according to the following procedure.

1 g of RG504H and 20 mg of octreotide were homogeneously dissolved in 20 ml of glacial acetic acid. The resulting solution was supplied to a spray drier at a flow rate of 2 ml/min through an ultrasonic nozzle (Sono-Tek, 8700-60 MS) that is a general single-feed type, sprayed in a spray drier (Kwangjin Corporation, Korea), and dried with dry air at 105° C., thereby yielding microspheres. The final microspheres were 30.7 μm in diameter, on average.

### TEST EXAMPLE 1

### In vitro Drug Release Tests of the Octreotide-Loaded Microspheres

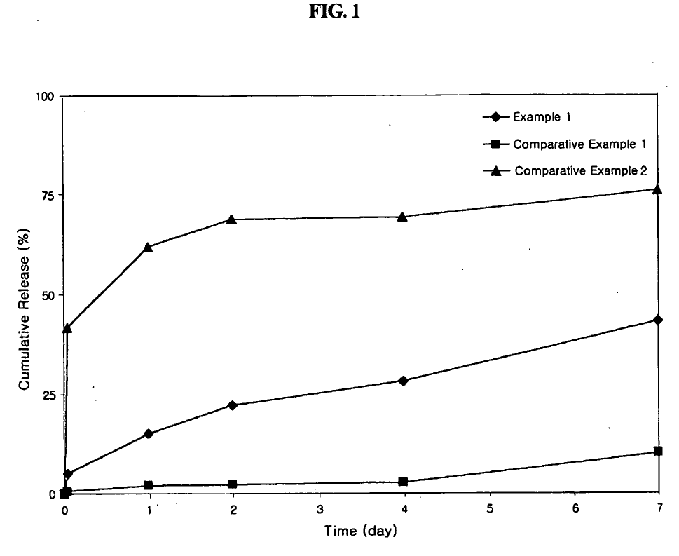

In vitro drug release profiles of the microsphere formulations were examined using 5 mg/ml of a microsphere formulation and 50 mM sodium acetate (pH 4.0) at 37° C. Released amounts of a drug from the microspheres were measured using a UV absorption spectrophotometer (280 nm) and a fluorescence detector (Ex: 280 nm; Em: 350 nm). In vitro drug release tests were carried out for the three microsphere formulations prepared in Example 1 and Comparative Examples 1 and 2, and the results are given in FIG. 1.

As shown in FIG. 1, microspheres (Comparative Example 1), prepared using RG502H, a hydrophilic polymer with a relatively low molecular weight, by spraying through a conventional single-feed nozzle, displayed a low initial release rate and a low continuous release rate of octreotide. Microspheres (Comparative Example 2), prepared using RG504H having a molecular weight higher than RG502H, showed a high initial release rate. In contrast, in the case of the microspheres (Example 1) prepared according to the present invention, including an inner core formed using RG502H, a hydrophilic polymer having a relatively high degradation rate, and an outer shell coating the inner core, formed using RG504H, having a higher molecular weight and a lower degradation rate than RG502H, the initial release of octreotide remarkably decreased, and was followed by a continuous release at a constant rate.

### EXAMPLE 2

### Preparation of Leuprolide-Loaded Microspheres using a Dual-Feed Nozzle

Microspheres were prepared to contain leuprolide as a drug in a final concentration of 10 wt % using biodegradable polymers, RG503H and R202H, according to the following procedure.

A solution A, to be supplied through an internal channel of a dual-feed nozzle, was prepared by homogeneously dissolving 0.44 g of the biodegradable polymer R202H and 60 mg of leuprolide in 10 ml of glacial acetic acid. A solution B, to be supplied through an external channel of the dual-feed nozzle, was prepared by homogeneously dissolving 0.46 g of the biodegradable polymer RG503H and 40 mg of leuprolide in 10 ml of glacial acetic acid. The two solutions were supplied to a spray drier at a flow rate of 1 ml/min respectively through internal and external channels of an ultrasonic dual-feed nozzle (Sono-Tek, 8700-25MS), sprayed in the spray drier (Kwangjin Corporation, Korea), and dried with dry air at 105° C., thereby yielding microspheres. The final microspheres were 29.8 μm in diameter, on average.

Microspheres, prepared in the following Examples and Test Examples by spray-drying protein-containing solutions with different compositions through a dual-feed nozzle, were found to effectively control the initial release and continuous release of a drug.

### EXAMPLE 3

### Preparation of BSA-Loaded PLGA Microspheres using a Dual-Feed Nozzle

According to the compositions summarized in Table 1, below, a suspension A and a solution B to be supplied respectively through internal and external channels of a dual-feed nozzle were prepared using biodegradable polymers and a protein drug. RG502H and RG504H biodegradable polymers were used, and bovine serum albumin (BSA) was used as the protein drug. Polyethylene glycol (PEG) having a molecular weight of 10,000 was used as an additive.

The suspension A and solution B were prepared as follows. Corresponding biodegradable polymers and additive were homogeneously dissolved in 10 ml of acetonitrile. In the resultant suspension A, BSA microparticles (average particle diameter: 2.3 μm) were suspended, thereby generating a final suspension A.

The two liquids were supplied to a spray drier at a flow rate of 1 ml/min respectively through internal and external channels of an ultrasonic dual-feed nozzle (Sono-Tek, 8700-25MS), sprayed in the spray drier (Kwangjin Corporation, Korea), and dried with dry air at 100° C., thereby yielding microspheres. The final microspheres were 31.5 μm in diameter, on average.

TABLE 1

| | | | | Composition of |
|---|---|---|---|---|
| | Composition of Suspension A | | | Solution B |
| Example | Polymer | Protein drug | Additive | Polymer |
| E. 3-1 | 0.4 g RG502H | 100 mg BSA | — | 0.5 g RG504H |
| E. 3-2 | 0.4 g RG502H | 100 mg BSA | 20 mg PEG | 0.5 g RG504H |

### COMPARATIVE EXAMPLE 3

### Preparation of BSA-Loaded RG502H Microspheres using a Single-Feed Nozzle

Microspheres were prepared to contain bovine serum albumin (BSA) as a protein drug in a final concentration of 10 wt % using a biodegradable polymer, RG502H, according to the following procedure.

0.9 g of RG502H was homogeneously dissolved in 20 ml of acetonitrile. 0.1 g of BSA microparticles (average particle diameter: 2.3 μm) was suspended in the resulting solution. The suspension was supplied to a spray drier at a flow rate of 2 ml/min through a general single-feed-type ultrasonic nozzle (Sono-Tek, 8700-60MS), sprayed in the spray drier (Kwangjin Corporation, Korea), and dried with dry air at 100° C., thereby yielding microspheres. The final microspheres were 30.9 pm in diameter, on average.

### COMPARATIVE EXAMPLE 4

### Preparation of BSA-Loaded RG504H Microspheres using a Single-Feed Nozzle

Microspheres were prepared to contain bovine serum albumin (BSA) as a protein drug in a final concentration of 10 wt % using a biodegradable polymer, RG504H, according to the following procedure.

0.9 g of RG504H was homogeneously dissolved in 20 ml of acetonitrile. 0.1 g of BSA microparticles (average particle diameter: 2.3 μm) was suspended in the resulting solution. The suspension was supplied to a spray drier at a flow rate of 2 ml/min through a general single-feed-type ultrasonic nozzle (Sono-Tek, 8700-60MS), sprayed in the spray drier (Kwangjin Corporation, Korea), and dried with dry air at 100° C., thereby yielding microspheres. The final microspheres were 32.3 μm in diameter, on average.

### COMPARATIVE EXAMPLE 5

### Preparation of BSA-Loaded PLGA Microspheres Coated with Water-Soluble Polymer using a Dual-Feed Nozzle

A suspension A and a solution B, to be supplied to a spray drier respectively through internal and external channels of a dual-feed nozzle, were prepared using biodegradable polymers and a protein drug. Microspheres were prepared using a water-insoluble polymer, RG502H, a water-soluble polymer, gelatin A, and bovine serum albumin (BSA) as the protein drug, according to the following procedure.

450 mg of the water-insoluble polymer RG502H was homogeneously dissolved in 15 ml of acetonitrile. 100 mg of BSA microparticles (average particle diameter: 2.3 μm) was suspended in the resulting solution, thereby generating a final suspension A. A solution B to be supplied through an external channel of a dual-feed nozzle was prepared by homogeneously dissolving 450 mg of gelatin A in 15 ml of purified water.

The two liquids were supplied to a spray drier at a flow rate of 1 ml/min respectively through internal and external channels of an ultrasonic dual-feed nozzle (Sono-Tek, 8700-25MS), sprayed in the spray drier (Kwangjin Corporation, Korea), and dried with dry air at 110° C., thereby yielding microspheres. The final microspheres were 30.1 μm in diameter, on average.

### TEST EXAMPLE 2

### In vitro Drug Release Tests of the Protein-Loaded Microspheres

In vitro drug release profiles of the protein drug-loaded microsphere formulations were examined using 5 mg/ml of a microsphere formulation and 33 mM phosphate buffer (pH 7.4) at 37° C. Cumulative amounts of the drug released from the microspheres were measured using a fluorescence detector (Ex: 280 nm; Em: 350 nm). In vitro drug release tests were carried out for the five microsphere formulations prepared in Example 3 and Comparative Examples 3, 4 and 5, and the results are given in Table 2, below.

TABLE 2

Cumulative drug release (%)

| Microsphere formulations | 1 hr | 24 hrs |
|---|---|---|
| E. 3-1 | 15.5 | 31.0 |
| E. 3-2 | 9.9 | 49.3 |
| C.E. 3 | 77.1 | 85.5 |
| C.E. 4 | 79.8 | 92.3 |
| C.E. 5 | 76.2 | 95.1 |

As shown in Table 2, in the case of microspheres (Comparative Examples 3 and 4) prepared by a conventional spray-drying method using a single-feed nozzle, regardless of the type of polymers used, most entrapped bovine serum albumin was released at the initial phase. Microspheres (Comparative Example 5) coated with the water-soluble polymer gelatin A using a dual-feed nozzle also displayed a high initial release rate of the entrapped protein. In contrast, in the case of the microspheres (Examples 3-1 and 3-2) prepared according to the present invention, including an inner core formed using RG502H and containing 10 wt % of BSA and an outer shell of RG504H coating the inner core, the initial release of BSA remarkably decreased. Also, the microspheres of the present invention showed a cumulative release rate lower than 50% for a 24-hr period, thereby providing the prolonged release of a drug.

Compared to microsphere formulations having a single polymer composition, prepared according to a conventional method using a single-feed nozzle, the microsphere formulations of the present invention, prepared by spraying two polymers with different physicochemical properties through a dual-feed nozzle to provide microspheres comprising a coated core, remarkably reduced the initial release of a drug while prolonging the drug release, thereby providing a desired release pattern for a drug.

### INDUSTRIAL APPLICABILITY

As described hereinbefore, the present invention provides a one-step method of preparing sustained release microspheres containing a drug encapsulated in a biodegradable polymer carrier using a spray drier. The present method is based on simultaneously spraying two liquids having different compositions using a single dual-feed nozzle and drying the sprayed droplets, thereby providing double-layered microspheres comprising a core of a first liquid coated with a film of a second liquid having a different composition. Polymeric microspheres prepared by the present method provide prolonged release of a drug for a predetermined period without a high initial release of the drug. Thus, the present method improves the disadvantages of conventional sustained release microsphere formulations, that is, a high initial drug release or a drug release that sharply decreases or increases with the passage of time, thereby making it possible to easily achieve desired release patterns for drugs.

1. A method of preparing sustained release microspheres encapsulating a drug in a biodegradable polymer carrier, comprising:
(a) preparing two different liquids for preparation of the sustained release microspheres comprising a biodegradable polymer, a drug, an additive and a solvent with different compositions for one or more of the components;
(b) simultaneously spraying the two different liquids through internal and external channels of an ultrasonic dual-feed nozzle, wherein one liquid is supplied through the internal channel and another liquid is supplied through the external channel, and the liquid supplied through the external channel does not contain water; and
(c) evaporating the solvent using dry air to dry sprayed droplets.

2. The method as set forth in claim 1, wherein the biodegradable polymer is selected from the group consisting of polylactide, polyglycolide, poly(lactide-co-glycolide), poly(lactide-co-glycolide)-glucose, polyorthoesters, polyanhydrides, polyamino acids, polyhydroxybutyric acid, polycaprolactone, polyalkylcarbonate, lipids, fatty acids, waxes and mixtures thereof.

3. The method as set forth in claim 2, wherein the biodegradable polymer is selected from polylactide and poly(lactide-co-glycolide).

4. The method as set forth in claim 1, wherein the drug is selected from peptides and proteins.

5. The method as set forth in claim 4, wherein the drug is selected from octreotide, luteinizing hormone releasing hormone (LHRH) analogs and salts thereof.

6. The method as set forth in claim 5, wherein the LHRH analogs are selected from triptorelin, leuprolide, goserelin, nafarelin, buserelin, histerelin and salts thereof.

7. The method as set forth in claim 5, wherein the salt of the drug is acetate.
